(19) United States
(12) Patent Application Publication
(10) Pub. No.: US 2022/0304842 A1
(43) Pub. Date: Sep. 29, 2022

(54) FLUID CONTROL SYSTEM FOR AN IMPLANTABLE INFLATABLE DEVICE

(71) Applicant: Boston Scientific Scimed, Inc., Maple Grove, MN (US)

(72) Inventors: Daragh Nolan, Co. Waterford (IE); Brian P. Watschke, Minneapolis, MN (US); Noel Smith, Windgap (IE); Moira B. Sweeney, St. Paul, MN (US); Peter Fewer, Co. Tipperary (IE); Thomas Sinnott, Wexford (IE); Richard Percy, Co. Cork (IE); Natalie Ann Borgos, Roseville, MN (US); Eduardo Marcos Larangeira, Clonmel (IE); John Gildea, Kildare (IE); Evania Ann Mareena, Clonmel (IE)

(21) Appl. No.: 17/655,958

(22) Filed: Mar. 22, 2022

Related U.S. Application Data
(60) Provisional application No. 63/200,739, filed on Mar. 25, 2021.

(51) Int. Cl. A61F 5/41; A61F 2/00

(52) U.S. Cl. CPC A61F 5/41; A61F 2/004; A61F 2005/415; A61F 2250/0002; A61F 2250/0096; A61F 2250/008; A61F 2210/009

(57) ABSTRACT

An implantable fluid operated device may include a fluid reservoir configured to hold fluid, an inflatable member, and a pump assembly configured to transfer fluid between the fluid reservoir and the inflatable member. The pump assembly may include one or more fluid pumps and one or more valves. An electronic control system may control operation of the pump assembly based on fluid pressure measurements and/or fluid flow measurements received from the one or more sensing devices. The electronic control system may include an internal component installed with the implanted device, and an external component that is operable by a user to provide user input, and to receive output from the implanted device.

### CROSS-REFERENCE TO RELATED APPLICATION

This application claims priority to U.S. Provisional Patent Application No. 63/200,739, filed on Mar. 25, 2021, entitled “FLUID CONTROL SYSTEM FOR AN IMPLANTABLE INFLATABLE DEVICE”, the disclosure of which is incorporated by reference herein in its entirety.

### TECHNICAL FIELD

This disclosure relates generally to bodily implants, and more specifically to bodily implants including a pump.

### BACKGROUND

Active implantable fluid operated devices often include one or more pumps that regulate a flow of fluid between different portions of the implantable device. One or more valves can be positioned within fluid passageways of the device to direct and control the flow of fluid so as to achieve inflation, deflation, pressurization, depressurization, Activation, deactivation and the like of different fluid filled implant components of the device. In some implantable fluid operated devices, sensors can be used to monitor fluid pressure and/or fluid volume within fluid passageways of the device. Accurate monitoring of conditions within the device, including pressure monitoring and flow monitoring, may provide for improved control of device operation, improved diagnostics, and improved efficacy of the device. In addition, sensors could be used to monitor external conditions from the device, including acceleration, angle, barometric pressure and temperature, which may facilitate the determination of operating modes of the device.

### SUMMARY

In a general aspect, an implantable fluid operated inflatable device includes a fluid reservoir; an inflatable member; and an electronic fluid control system coupled between the fluid reservoir and the inflatable member and configured to control fluid between the fluid reservoir and the inflatable member. The electronic fluid control system may include a housing; a fluid control system received in the housing, including fluidic architecture including at least one valve and at least one pump positioned in a fluid passageway within in the housing; and an electronic control system received in the housing, the electronic control system including at least one processor configured to control operation of the at least one pump and at least one valve; and a communication module configured to communicate with at least one external device. The implantable fluid operated inflatable device may also include at least one pressure sensing device configured to sense a fluid pressure in the implantable fluid operated inflatable device, and to transmit the sensed pressure to the electronic control system.

In some implementations, the reservoir is bonded to an outer surface of the housing. In some implementations, the reservoir includes a bellows structure configured to contract as fluid is expelled from the reservoir, and to expand as fluid flows into the reservoir. In some implementations, the reservoir is received within the housing. In some implementations, a closed bellows is provided within the housing, wherein the closed bellows is filled with a compressible fluid, such that the closed bellows is configured to contract in response to expansion of the reservoir, and to expand in response to contraction of the reservoir.

In some implementations, the electronic control system is configured to receive a user input from the external device, and to control operation of the at least one pump and the at least one valve in response to the received user input. In some implementations, the electronic control system is configured to adjust operation of the at least one pump and the at least one valve to reduce a pressure at the inflatable member and initiate deflation of the inflatable member in response to detection of a signal generated by interaction of a magnet with the electronic control system, positioned corresponding to the fluid-controlled inflatable device for a preset period of time. In some implementations, the electronic control system is configured to control operation of the at least one pump and the at least one valve in response to user inputs including at least one of a fluctuation in pressure detected by the at least one sensing device in response to a tapping input or a tugging input; or a motion event detected by a motion detecting device of the fluid operated inflatable device or the external device. The tapping input may include a series of taps in a preset sequence detected by a piezoelectric element of the at least one pump or the at least one valve. The preset sequence may include a wake-up sequence to wake the fluid operated inflatable device, including a first tapping sequence defined by a first number of taps in a first pattern; and an activation sequence corresponding to a user input, including a second tapping sequence defined by a second number of taps in a second pattern.

In some implementations, the electronic control system is configured to monitor pressure levels in the fluid-controlled inflatable device, and to control operation of the at least one pump and the at least one valve in response to detected fluctuations in pressure, including control the at least one pump and the at least one valve to reduce a pressure at the inflatable member and deflate the inflatable member in response to detection of the inflatable member in an inflated state for greater than a preset period of time; control the at least one pump and the at least one valve to maintain a current state of the fluid-controlled inflatable device in response to detection of a spike in pressure having a duration that is less than a preset period of time; and control the at least one pump and the at least one valve to maintain the current state of the fluid-controlled inflatable device in response to detection of a change in atmospheric conditions.

In some implementations, the electronic control system is configured to detect a failure in the fluid-controlled inflatable device in response to detection of a time to reach a set pressure exceeding a set period of time or an inability to reach the set pressure; output an alert of the detected failure to the external device; and isolate fluid from an area of the detected failure.

In some implementations, the at least one pump includes a first piezoelectric pump in a first fluid channel of the fluidic architecture and a second piezoelectric in a second fluid channel of the fluidic architecture, In a deflation mode, the first piezoelectric pump is configured to operate to pump fluid from the inflatable member to the reservoir, while the second piezoelectric pump is in a standby mode, and vibration generated by operation of the first piezoelectric pump may be harvested by the second piezoelectric pump in the standby mode for conversion to energy. In an inflation mode, the second piezoelectric pump is configured to operate to pump fluid from the reservoir to the inflatable member, while the first piezoelectric pump is in the standby mode, and vibration generated by operation of the second piezoelectric pump may be harvested by the first piezoelectric pump in the standby mode for conversion to energy. In a standby mode of the fluid operated inflatable device in which the first piezoelectric pump and the second piezoelectric pump are both in the standby mode, vibration generated due to motion of a patient in which the fluid operated inflatable device is implanted may be harvested by the first piezoelectric pump and the second piezoelectric pump for conversion to energy.

In some implementations, the fluidic architecture includes a first uni-directional pump and a first passive valve positioned in a first fluid passageway to selectively generate and control fluid flow in a first direction, from the inflatable member toward the reservoir; a second uni-directional pump and a second passive valve positioned in a second fluid passageway to selectively generate and control fluid flow in a second direction, from the reservoir to the inflatable member; a first sensing device positioned to sense a fluid pressure at the reservoir; a second sensing device positioned to sense a fluid pressure at the inflatable member; and an active valve positioned in-line with the inflatable member. In a first mode, the active valve may be configured to be closed by the electronic control system in response to detection of a pressure spike at the inflatable member to prevent deflation of the inflatable member. In a second mode, the active valve may be configured to be opened by the electronic control in response to detection of a power loss to the electronic fluid control system to allow deflation of the inflatable member.

In some implementations, the fluidic architecture includes a first uni-directional pump positioned in a first fluid passageway and configured to generate a flow of fluid in a first direction, from the inflatable member toward the reservoir; a second uni-directional pump positioned in a second fluid passageway and configured to generate a flow of fluid in a second direction, from the reservoir toward inflatable member; a first passive valve positioned in the first fluid passageway, between the first uni-directional pump and the reservoir so as to restrict fluid flow in the first direction in the first fluid passageway and to prevent back flow of fluid in the first fluid passageway while the second uni-directional pump is in an operational mode and the first uni-directional pump is in a standby mode; a second passive valve positioned in the second fluid passageway, between the second uni-directional pump and the reservoir so as to restrict fluid flow in the second direction in the second fluid passageway and to prevent back flow of fluid in the second fluid passageway while the first uni-directional pump is in an operational mode and the second uni-directional pump is in a standby mode; a first sensing device positioned to sense a fluid pressure at the reservoir; and a second sensing device positioned to sense a fluid pressure at the inflatable member.

In some implementations, the fluidic architecture includes a uni-directional pump positioned in a fluid passageway; a first active valve positioned in the fluid passageway, between the pump and the reservoir, and configured to be selectively activated by the electronic control system; a second active valve positioned in the fluid passageway, between the pump and the inflatable member, and configured to be selectively activated by the electronic control system; a third active valve positioned in a fluid passageway between the pump and the reservoir and configured to be selectively activated by the electronic control system; and a fourth active valve in a fluid passageway between the pump and the inflatable member and configured to be selectively activated by the electronic control system. In an inflation mode, the first active valve and the second active valve are opened by the electronic control system and the third active valve and the fourth active valve are closed by the electronic control system so that fluid is pumped from the reservoir to the inflatable member. In a deflation mode, the third active valve and the fourth active valve are opened by the electronic control system and the first active valve and the second active valve are closed by the electronic control system so that fluid is pumped from the inflatable member to the reservoir.

In some implementations, the fluidic architecture includes a first combined pump and valve device positioned in a first fluid passageway to selectively generate and control fluid flow in a first direction, from the inflatable member toward the reservoir; a first sensing device positioned to sense a fluid pressure at the reservoir; a second combined pump and valve device positioned in a second fluid passageway to selectively generate and control fluid flow in a second direction, from the reservoir toward the inflatable member; and a second sensing device positioned to sense a fluid pressure at the inflatable member.

In some implementations, the fluidic architecture includes a first piezoelectric pump and valve device positioned in a first fluid passageway, wherein the first piezoelectric pump and valve device is configured to selectively generate and control fluid flow in a first direction, from the inflatable member toward the reservoir, and to sense a fluid pressure at the reservoir; and a second piezoelectric pump and valve device positioned in a second fluid passageway, wherein the second piezoelectric pump and valve device is configured to selectively generate and control fluid flow in a second direction, from the reservoir toward the inflatable member, and to sense a fluid pressure at the inflatable member. In some implementations, the fluidic architecture includes a pump; a first three-way valve positioned between the pump and the reservoir, the first three-way valve having a first port thereof open to maintain fluidic communication with the pump; and a second three-way valve positioned between the pump and the inflatable member, the second three-way valve having a first port thereof open to maintain fluidic communication with the pump. In a deflation mode, a second port of the first three-way valve is opened and a third port of the first three-way valve is closed to direct fluid flow from first port to the second port of the first three-way valve, and a second port of the second three-way valve is opened and a third port of the second three-way valve is closed to direct fluid flow from the first port to the second port of the second three-way valve. In an inflation mode, the second port of the first three-way valve is closed and the third port of the first three-way valve is opened to direct fluid flow from first port to the third port of the first three-way valve, and the second port of the second three-way valve is closed and the third port of the second three-way valve is opened to direct fluid flow from the first port to the third port of the second three-way valve.

### BRIEF DESCRIPTION OF THE DRAWINGS

FIG. 1 is a block diagram of an implantable fluid operated inflatable device according to an aspect.

FIGS. 2A and 2B illustrate example implantable fluid operated inflatable devices according to an aspect.

FIG. 3 is a schematic diagram of a fluidic architecture of an implantable fluid operated inflatable device according to an aspect.

FIG. 4 is a schematic illustration of an example electronic fluid control system for an implantable fluid operated inflatable device.

FIG. 5 is a schematic illustration of a first example fluidic architecture of the example fluid control system shown in FIG. 4.

FIG. 6 is a schematic illustration of a second example fluidic architecture of the example fluid control system shown in FIG. 4.

FIG. 7 is a schematic illustration of a third example fluidic architecture of the example fluid control system shown in FIG. 4.

FIG. 8 is a schematic illustration of a fourth example fluidic architecture of the example fluid control system shown in FIG. 4.

FIG. 9 is a schematic illustration of a fifth example fluidic architecture of the example fluid control system shown in FIG. 4.

FIG. 10 is a schematic illustration of a sixth example fluidic architecture of the example fluid control system shown in FIG. 4

FIG. 11 is a schematic illustration of a seventh example fluidic architecture of the example fluid control system shown in FIG. 4.

FIGS. 12A-12C are schematic illustrations of example implantable fluid operated inflatable devices according to an aspect.

FIGS. 13A and 13B are schematic illustrations of example implantable fluid operated inflatable devices according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

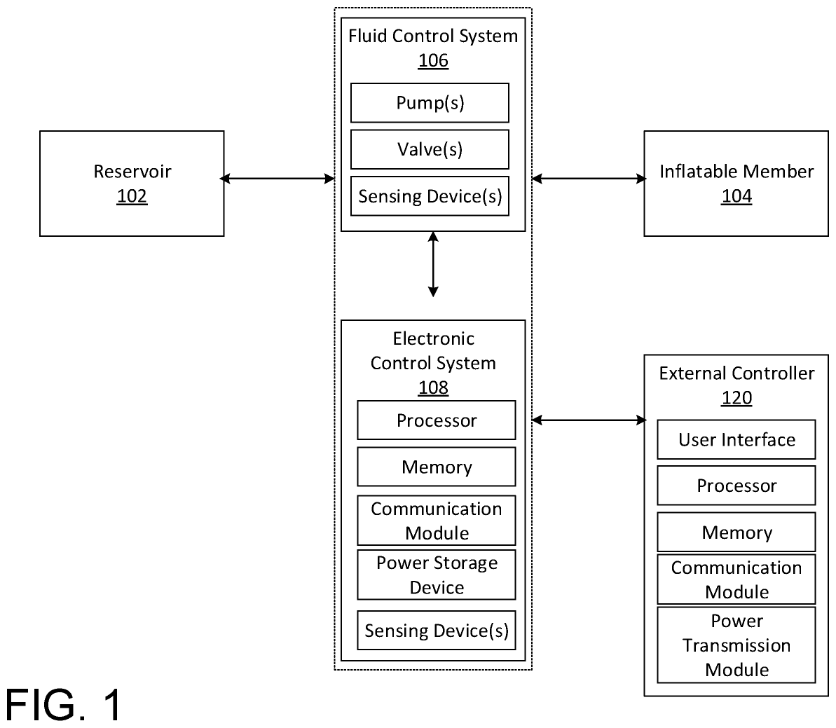

FIG. 1 is a block diagram of an example implantable fluid operated inflatable device 100. The example device 100 shown in FIG. 1 includes a fluid reservoir 102, an inflatable member 104, and a fluid control system 106 including fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics system of the device 100. In some implementations, the example device 100 includes an electronic control system 108. The electronic control system 108 may provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes, for example, a processor, a memory, a communication module, a power storage device, or battery, sensing devices such as, for example an accelerometer, and other such components configured to provide for the operation and control of the implantable fluid operated inflatable device 100. For example, the communication module may provide for communication with one or more external devices such as, for example, an external controller 120. The external controller 120 may be configured to receive user inputs through, for example, a user interface, and to transmit the user inputs, for example, through a communication module, to the electronic control system 108 for processing, operation and control of the device 100. The electronic control system 108 may, through the communication module, transmit operational information to the external controller 120. This may allow operational status of the inflatable device 100 to be provided, for example, through the user interface, to the user, diagnostics information to be provided to a physician, and the like. In some examples, the external controller 120 includes a power transmission module providing for charging of the components of the internal electronic control system 108. In some examples, transmission of power for the recharging of the internal electronic control system 108 is provided in an external device that is separate from the external controller 120. In some implementations the external controller 120 can include sensing devices such as a pressure sensor, an accelerometer and other such sensing devices. An external pressure sensor in the external controller 120 may provide, for example, a local atmospheric or working pressure to the internal electronic control system 108, to allow the inflatable device 100 to compensate for variations in pressure. An accelerometer in the external controller 120 may provide detected patient movement to the internal electronic control system 108 for control of the inflatable device 100. The fluid reservoir 102, the inflatable member 104, and the fluid control system 106 may be internally implanted into the body of the patient. In some implementations, the electronic control system 108 is coupled to or incorporated into a housing of the fluid control system 106. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable device 100. In some implementations, operation of the implantable fluid operated inflatable device 100 may be manually controlled.

In some examples, electronic monitoring and control of the fluid operated inflatable device 100 may provide for improved patient control of the device, improved patient comfort, and improved patient safety. In some examples, electronic monitoring and control of the fluid operated device 100 may afford the opportunity for tailoring of the operation of the device 100 by the physician without further surgical intervention. Fluidic architecture defining the flow and control of fluid through the fluid operated inflatable device 100, including the placement of fluidics components such as pumps, valves, sensing devices and the like, may allow the device 100 to effectively respond to user inputs, and to quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

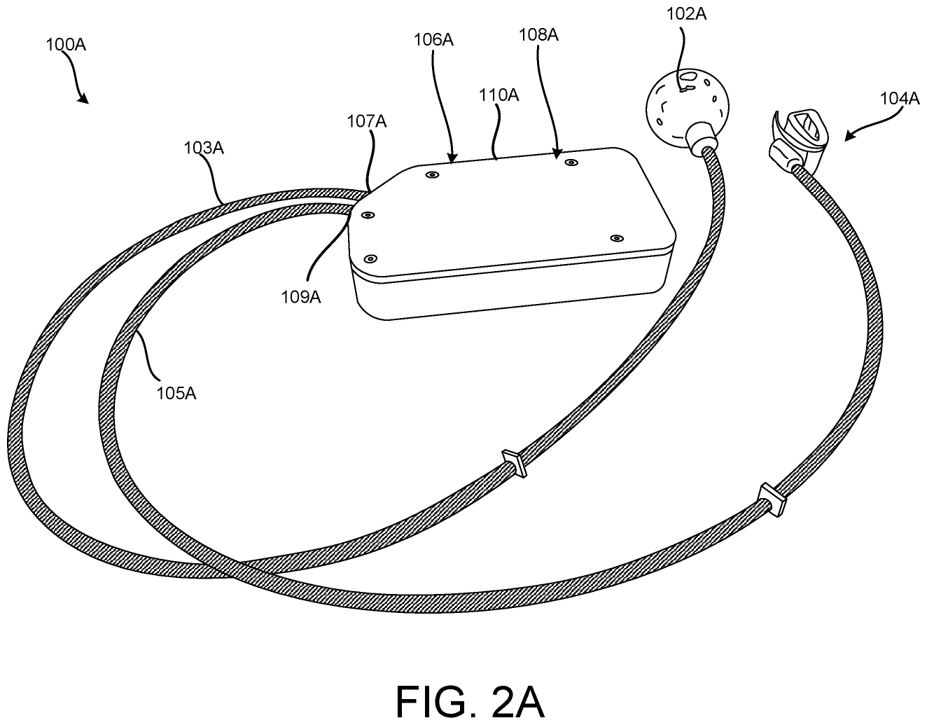

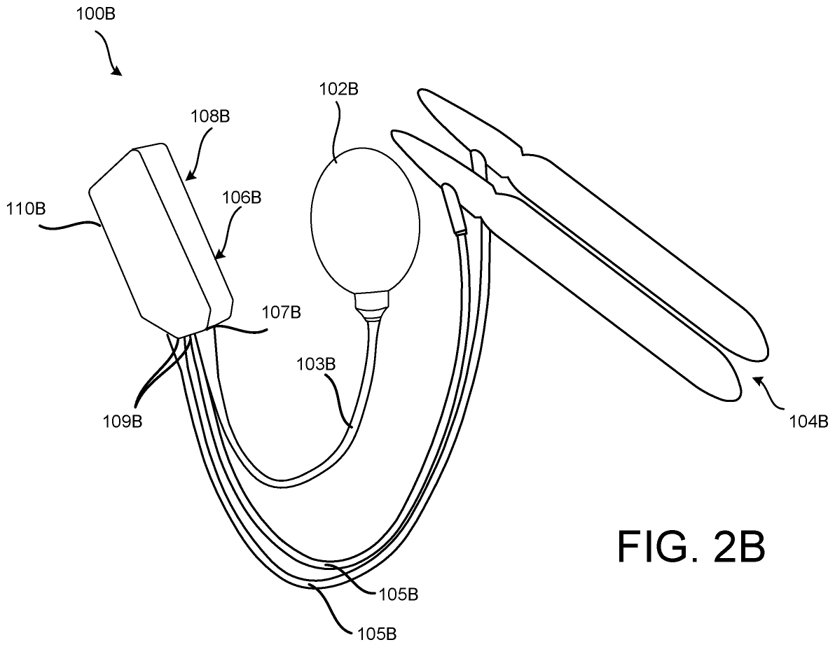

The example implantable fluid operated inflatable device 100 may be representative of a number of different types of implantable fluid operated devices. For example, the device 100 shown in FIG. 1 may be representative of an artificial urinary sphincter 100A as shown in FIG. 2A, an inflatable penile prosthesis 100B as shown in FIG. 2B, and other such implantable inflatable devices that rely on the control of fluid flow to components of the device to achieve inflation, pressurization, deflation, depressurization, deactivation and the like.

The example artificial urinary sphincter 100A shown in FIG. 2A includes a fluid control system 106A including fluidics components such as pumps, valves, sensing devices and the like positioned in fluid passageways, and an electronic control system 108A configured to provide for the transfer of fluid between a reservoir 102A and an inflatable cuff 104A via the fluidics components. Fluidics components of the fluid control system 106A, and electronic components of the electronic control system 108A may be received in a housing 110A. A first conduit 103A connects a first fluid port 107A of the fluid control system 106A/electronic control system 108A received in the housing 110A with the reservoir 102A. A second conduit 105A connects a second fluid port 109A of the fluid control system 106A/electronic control system 108A received in the housing 110A with the inflatable cuff 104A.

The example penile prosthesis 100B shown in FIG. 2B includes a fluid control system 106B including fluidics components such as pumps, valves, sensing devices and the like positioned in fluid passageways, and an electronic control system 108B configured to provide for the transfer of fluid between a fluid reservoir 102B and inflatable cylinders 104B via the fluidics components. Fluidics components of the fluid control system 106B, and electronic components of the electronic control system 108B may be received in a housing 110B. A first conduit 103B connects a first fluid port 107B of the fluid control system 106B/electronic control system 108B received in the housing 110B with the reservoir 102B. One or more second conduits 105B connect one or more second fluid ports 109B of the fluid control system 106A/electronic control system 108A received in the housing with the inflatable cylinders 104B.

The principles to be described herein may be applied to these and other types of implantable fluid operated inflatable devices that rely on a pump assembly including various fluidics components to provide for the transfer of fluid between the different fluid filled implantable components to achieve inflation, deflation, pressurization, depressurization, deactivation, occlusion and the like for effective operation. The example devices 100A, 100B shown in FIGS. 2A and 2B include electronic control systems 108A, 108B to provide for the monitoring and control of pressure and/or fluid flow through the respective devices 100A, 100B. Some of the principles to be described herein may also be applied to implantable fluid operated inflatable devices that are manually controlled.

As noted above with respect to FIG. 1, the fluid control system 106 can include a pump assembly including, for example, one or more pumps and one or more valves positioned within a fluid circuit of the pump assembly to control the transfer fluid between the fluid reservoir 102 and the inflatable member 104. In some examples, the pump(s) and/or the valve(s) are electronically controlled. In some examples, the pump(s) and/or the valve(s) are manually controlled. In some examples, the pump assembly includes a fluid manifold having fluidic channels formed therein, defining the fluid circuit. In an example in which the pump assembly is electronically powered and/or controlled, the manifold may be a hermetic manifold that can contain and segment the flow of fluid from electronic components of the pump assembly, to prevent leakage and/or gas exchange. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization and deactivation of the components of the implantable fluid operated device to provide for patient safety and device efficacy.

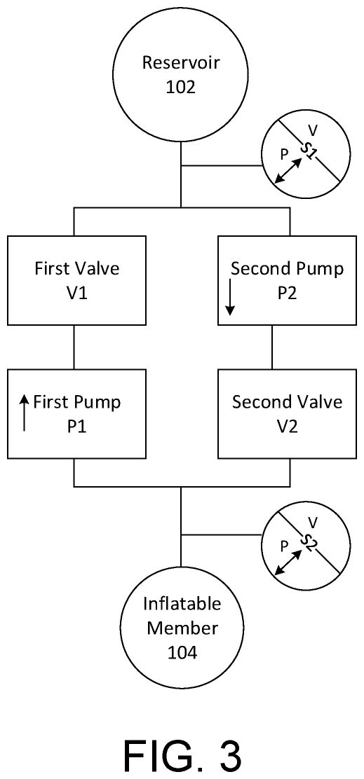

FIG. 3 is a schematic diagrams of an example fluidic architecture for an implantable fluid operated inflatable device, according to an aspect. The fluidic architecture of an implantable fluid operated inflatable device can include other orientations of fluidic channels, valve(s), pressure sensor(s) and other components than shown in FIG. 3. A fluidic architecture that can accommodate back pressure, pressure surges and the like enhances the performance, efficacy and efficiency of the fluid operated device 100.

The example fluidic architecture shown in FIG. 3 includes channels guiding the flow of fluid between the reservoir 102 and the inflatable member 104. In the example shown in FIG. 3, a first valve V1 in a first fluidic channel controls the flow of fluid, generated by a first pump P1, from the inflatable member 104 to the reservoir 102. A second valve V2 in a second fluidic channel controls the flow of fluid, generated by a second pump P2, from the reservoir 102 to the inflatable member 104. A first sensing device S1 senses a fluid pressure at the reservoir 102, and a second sensing device S2 senses a fluid pressure at the inflatable member 104. The first and second sensing devices S1, S2 may provide for the monitoring of fluid flow and/or fluid pressure in the fluidic channels. In the arrangement shown in FIG. 3, one of the first pump P1 or the second pump P2 is active, while the other of the first pump P1 or the second pump P2 is in a standby mode, such that the first and second pumps P1, P2 do not typically operate simultaneously. Operation of the first and second pumps P1, P2 and the first and second valves V1, V2 (between the open and closed states) may be controlled by the control system 108 as described above, based on conditions (for example, fluid pressure and/or fluid flow rate) in the first and second fluidics channels in the areas proximate the reservoir 102 and the inflatable member 104 sensed by the first and second sensing devices S1, S2.

For example, operation of the first pump P1 with the first valve V1 open (and with the second pump P2 in the standby mode and the second valve V2 closed) may provide for the deflation of the inflatable member 104. The first pump P1 continues to operate until a pressure sensed by the second sensing device S2 (which is in-line with the inflatable member 104) indicates that a desired state of deflation of the inflatable member 104 has been achieved (base on, for example, a fluid pressure sensed by the second sensing device S2). To maintain the deflated state, both the first and second pumps P1, P2 may be placed in the standby mode and both the first and second valves V1, V2 may be closed. Operation of the second pump P2 with the second valve V2 open (and with the first pump P1 in the standby mode and the first valve V1 closed) may provide for the inflation of the inflatable member 104. The second pump P2 continues to operate until a pressure sensed by the first sensing device S1 indicates a desired state of inflation of the inflatable member 104 has been achieved (based on, for example, a fluid pressure sensed by the first sensing device S2). To maintain the inflated state, both the first and second pumps P1, P2 may be placed in the standby mode and both the first and second valves V1, V2 may be closed. The valves V1, V2 may provide for the selective sealing of the respective fluidic channel(s) so as to maintain a set state of the fluid operated device. Interaction with the valves V1, V2 (and the corresponding change in fluid flow through the fluidic architecture of the device) may change the set state of the fluid operated device. Valves V1, V2 that maintain the set state of the device until the patient requires a change in the set state of the device and initiates the required change in the set state of the device provide enhanced patient safety and improved device efficacy.

In some examples, one or more of the valves included in the fluidic architecture are normally open valves. Normally open valves default to an open state, and close (and remain closed) in response to the application of power. The use of normally open valves in the example arrangement shown in FIG. 3 may provide failsafe measures in the event of, for example, power failure or other system failure which would result in the loss of control of the pumps P1, P2 and/or valves V1, V2. For example, a loss of power (or other system failure) that results in this type of loss of control, in state in which the inflatable member 104 is inflated, the valves V1, V2 are closed and the pumps P1, P2 are in the standby state, could cause patient discomfort and/or compromise patient safety. The use of normally open valves in the fluidics architecture allows for the valves V1, V2 to open in the event of a power loss, pressure to be relieved from the inflatable member 104, and for the fluid in the system to reach equilibrium.

In some examples, one or more of the valves included in the fluidic architecture may be normally closed valves, which default to the closed state, and open (and remain open) in response to the application of power. Normally closed may not provide the failsafe measures described above, depending on a position of the normally closed valve in the fluidic architecture. However, the use of one or more normally closed valves in the fluidic architecture may reduce power consumption of the fluid operated inflatable device 100. Many of the valves included in the fluidic architecture remain in the closed state for considerably more time than they are in the open state (for example, to maintain a current state of the fluid operated inflatable device 100). Because normally closed valves default to the closed state and do not rely on the application of power to remain in the closed state, the use of one or more normally closed valves the fluidic architecture may reduce power consumption (when compared to the use of normally open valves). This may increase longevity of the fluid operated inflatable device 100, reduce physician intervention required for continued operation (to, for example, replace power cells), and/or reduce re-charging requirements and/or increase intervals between re-charging.

Power consumption can be reduced through the passive movement of fluid through the fluidic channels of the fluid operated inflatable device 100, to reduce an amount of pumping needed to achieve a desired level of deflation of the inflatable member 104. For example, in the inflated state, a pressure at the inflatable member 104 is greater than a pressure at the reservoir 102. In the example arrangement shown in FIG. 3, to achieve a desired level of deflation of the inflatable member 104, the first valve V1 may be opened (without activation of the first pump P1, and with the second valve V2 closed and the second pump P2 in standby mode) to allow fluid to naturally flow out of the inflatable member 104. The first pump P1 can be activated to relieve any residual pressure not relieved by the passive flow of fluid out of the inflatable member 104 in this manner, based on a fluid pressure sensed by the first and/or second sensing devices S1, S2.

In some examples, pressure sensing devices (such as the sensing devices S1, S2 illustrated in the example fluidic architecture shown in FIG. 3) can support various different ways of regulating, measuring and controlling pressure in the fluidics architecture of the fluid operated inflatable device 100, and can be positioned so as to provide for monitoring of a fluid pressure at the reservoir 102 and at the inflatable member 104. For examples, the sensing devices S1, S2 (and/or other pressure sensing devices) may be positioned so as to detect surges or spikes in fluid pressure at various locations within the fluid operated inflatable device 100, and to control the pumps P1, P2 and valves V1, V2 accordingly, to maintain a current state of the fluid operated inflatable device 100 and/or to provide for patient comfort and safety.

For example, the fluid reservoir 102A of the example artificial urinary sphincter 100A described above with respect to FIG. 2A in placed intra-abdominally in the patient. A pressure sensing device (such as the first sensing device S1) positioned at the fluid reservoir 102A could thus provide an indication of abdominal pressure. If a spike or surge in pressure is detected by the first sensing device S1 (due to, for example, physical activity, an impact, a fall and the like), the system can respond by, for example, increasing a pressure at the inflatable cuff 104A, and the patient can retain continence through the pressure spike. In an example including the first sensing device S1 at the reservoir 102A and the second sensing device S2 at the inflatable cuff 104A, pressure measurements taken by each of the sensors S1, S2 can be used to determine, for example, how much pressure is required at the inflatable cuff 104A to counteract the spike in pressure at the reservoir 102A.

As noted above, in the inflated state, the pressure at the inflatable member 104 is greater than the pressure at the reservoir 102. The pressure differential between the inflatable member 104 and the reservoir 102 can be used for passive deflation of the inflatable member 104. As fluid in the inflatable device 100 reaches equilibrium, measurements from the sensing devices S1, S2 positioned as shown in the example fluidics architecture of FIG. 3, at the reservoir 102 and the inflatable member 104, can be used to determine when to engage the first pump P1 to maximize energy conservation, while also managing the time to transition from the inflated state to the desired level of deflation of the inflatable member 104. In some examples, positioning of the first and second sensing devices S1, S2 as shown may provide detection of blockages, slow leaks and the like within the fluidics architecture, and may allow the system to operate the pumps P1, P2 and valves V1, V2 to compensate for the detected fault.

In the example arrangement shown in FIG. 3, to achieve a desired level of deflation of the inflatable member 104, the first valve V1 may be opened (without activation of the first pump P1, and with the second valve V2 closed and the second pump P2 in standby mode) to allow fluid to naturally flow out of the inflatable member 104. The first pump P1 can be activated to relieve any residual pressure not relieved by the passive flow of fluid out of the inflatable member 104 in this manner, based on a fluid pressure sensed by the first and/or second sensing devices S1, S2

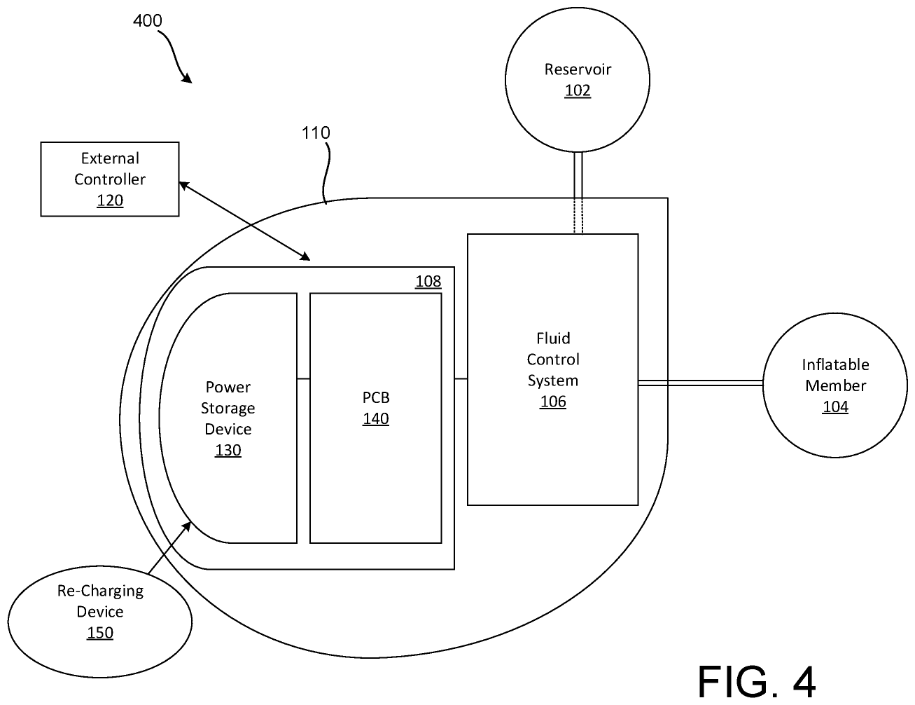

FIG. 4 is a schematic illustration of an example electronic fluid control system 400 for an implantable fluid operated inflatable device, according to an aspect. In some examples, the electronic fluid control system 400 provides for the transfer of fluid between the reservoir 102 and the inflatable member 104, and for the monitoring and control of components of the fluidics architecture within the fluid control system 106. In some example, the electronic control system 108 controls the operation of the components of the fluidics architecture of the fluid control system 106. In some examples, the electronics control system 108 includes a printed circuit board (PCB) 140. In some examples, the PCB 140 includes a processor, a memory, a communication module, sensing devices, and other such components. In some examples, the electronic control system 108 can communicate with the external controller 120 to, for example, receive user inputs, output information to the user and the like. In some examples, the control system 108 includes the power storage device 130, or battery 130 that provides power for operation of the components of the electronic control system 108 and for operation of the components of the fluid control system 106. In some example, the power storage device 130 can be re-charged by, for example, an external re-charging device 150. In some examples, the fluid control system 106 and components thereof, and the electronic control system 108 and components thereof are received in the housing 110.

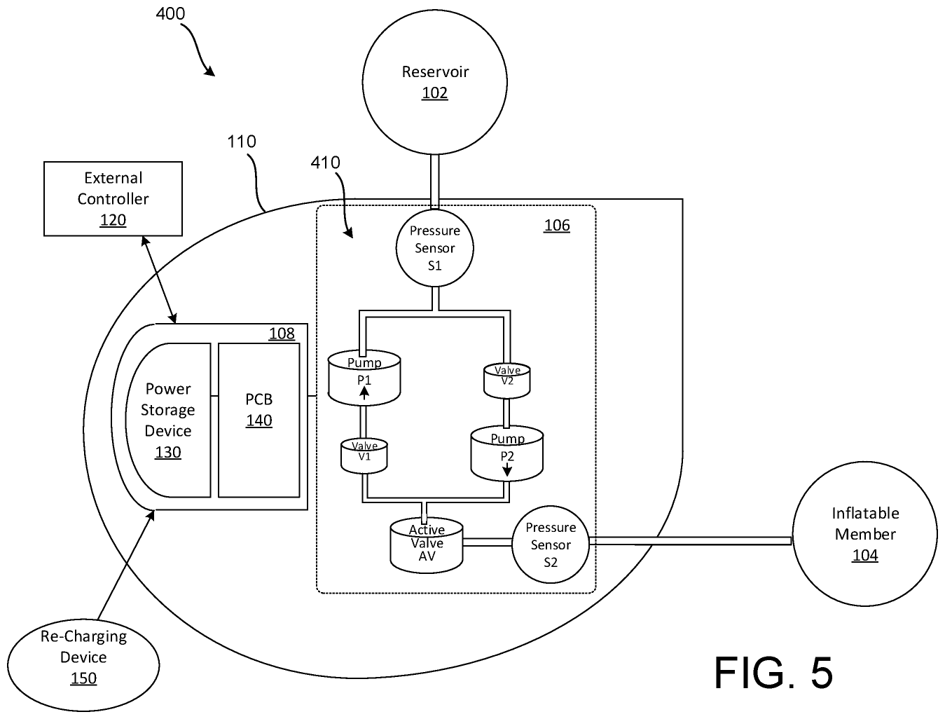

FIG. 5 illustrates the example electronic fluid control system 400 including the fluid control system 106 having a first example fluidic architecture 410. The first example fluidic architecture 410 includes the first pump P1 and the first valve V1 controlling the flow of fluid in a first direction, from the inflatable member 104 to the reservoir 102, and the second pump P2 and the second valve V2 controlling the flow of fluid in a second direction, from the reservoir 102 to the inflatable member 104. In the first example fluidic architecture 410 shown in FIG. 5, the first pump P1 is a uni-directional pump, and the first valve V1 is a passive check valve that restricts flow in the first fluidic channel and allows flow only in the first direction. The second pump P2 is a uni-directional pump, and the second valve V2 is a passive check valve that restricts flow in the second fluidic channel and allows flow only in the second direction. The first sensing device S1 is positioned to sense a fluid pressure at the reservoir 102, and the second sensing device S2 is positioned to sense a fluid pressure at the inflatable member 104. The first and second passive check valves V1, V2 arranged as shown relative to the first and second pumps P1, P2 prevent back flow of fluid through the pumps P1, P2. The first example fluidic architecture 410 includes an active valve AV positioned in-line with the inflatable member 104. The active valve AV positioned as shown may prevent fluid from leaking from the inflatable member 104 back through the first pump P1 and unintentionally deflating the inflatable member 104, for example, in response to a sudden spike in pressure at the inflatable member 104 due to an impact, physical exertion, a fall and the like.

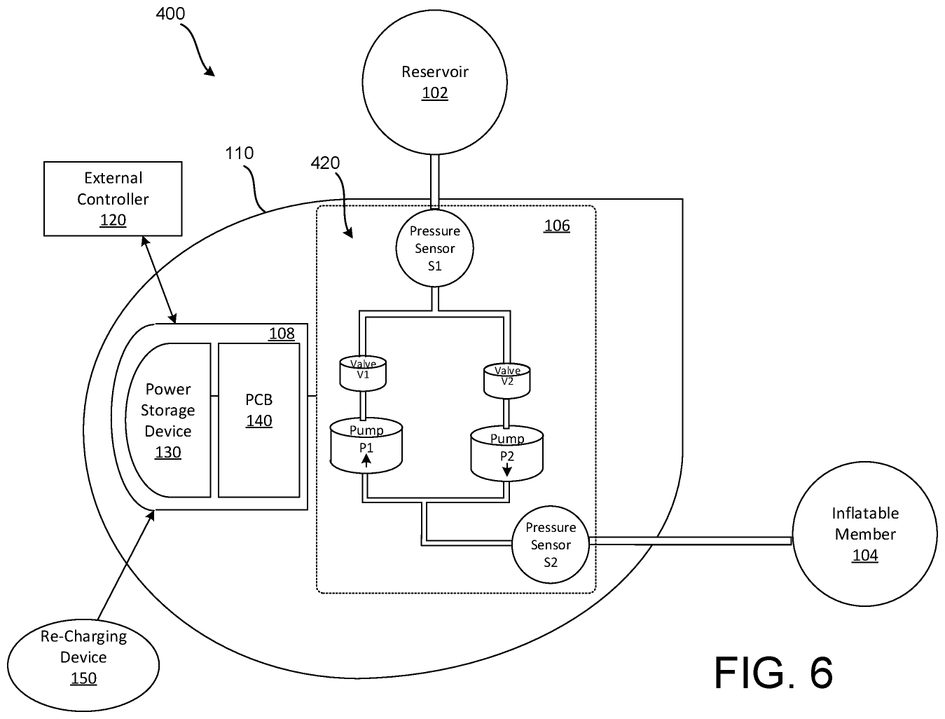

FIG. 6 illustrates the example electronic fluid control system 400 including the fluid control system 106 having a second example fluidic architecture 420. The second example fluidic architecture 420 includes the first pump P1 and the first valve V1 controlling the flow of fluid in a first direction, from the inflatable member 104 to the reservoir 102, and the second pump P2 and the second valve V2 controlling the flow of fluid in a second direction, from the reservoir 102 to the inflatable member 104. In the second example fluidic architecture 420 shown in FIG. 6, the first pump P1 is a uni-directional pump, and the first valve V1 is a passive check valve that restricts flow in the first fluidic channel and allows flow only in the first direction. The second pump P2 is a uni-directional pump, and the second valve V2 is a passive check valve that restricts flow in the second fluidic channel and allows flow only in the second direction. The first sensing device S1 is positioned to sense a fluid pressure at the reservoir 102, and the second sensing device S2 is positioned to sense a fluid pressure at the inflatable member 104. The first passive check valve V1 arranged as shown relative to the first pump P1 prevents back flow of fluid through the first pump P1 and inadvertent flow of fluid from the inflatable member 104 to the reservoir 102. The second passive check valve V2 arranged as shown prevents back flow of fluid through the second pump P2.

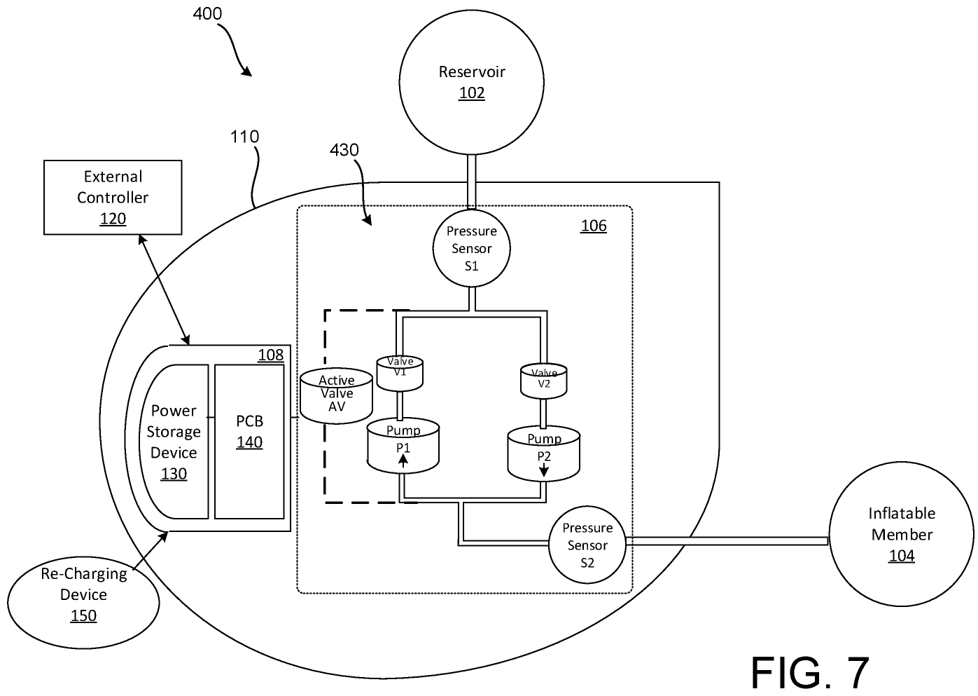

FIG. 7 illustrates the example electronic fluid control system 400 including the fluid control system 106 having a third example fluidic architecture 430. The third example fluidic architecture 430 includes the first pump P1 and the first valve V1 controlling the flow of fluid in a first direction, from the inflatable member 104 to the reservoir 102, and the second pump P2 and the second valve V2 controlling the flow of fluid in a second direction, from the reservoir 102 to the inflatable member 104. In the third example fluidic architecture 430, the first pump P1 is a uni-directional pump, and the first valve V1 is a passive check valve that restricts flow in the first fluidic channel and allows flow only in the first direction. The second pump P2 is a uni-directional pump, and the second valve V2 is a passive check valve that restricts flow in the second fluidic channel and allows flow only in the second direction. The first sensing device S1 is positioned to sense a fluid pressure at the reservoir 102, and the second sensing device S2 is positioned to sense a fluid pressure at the inflatable member 104. The first and second passive check valves V1, V2 arranged as shown relative to the first and second pumps P1, P2 prevent back flow of fluid through the pumps P1, P2. The third example fluidic architecture 430 includes an active valve AV that is positioned to act as a failsafe in the event of a loss of power. In the arrangement of components shown in the third example fluidic architecture, the active valve AV may be a normally open valve. In the event of a power loss to the electronic fluid control system 400, the active valve AV will open and allow the inflatable member 104 to de-pressurize, thus providing for patient comfort and safety.

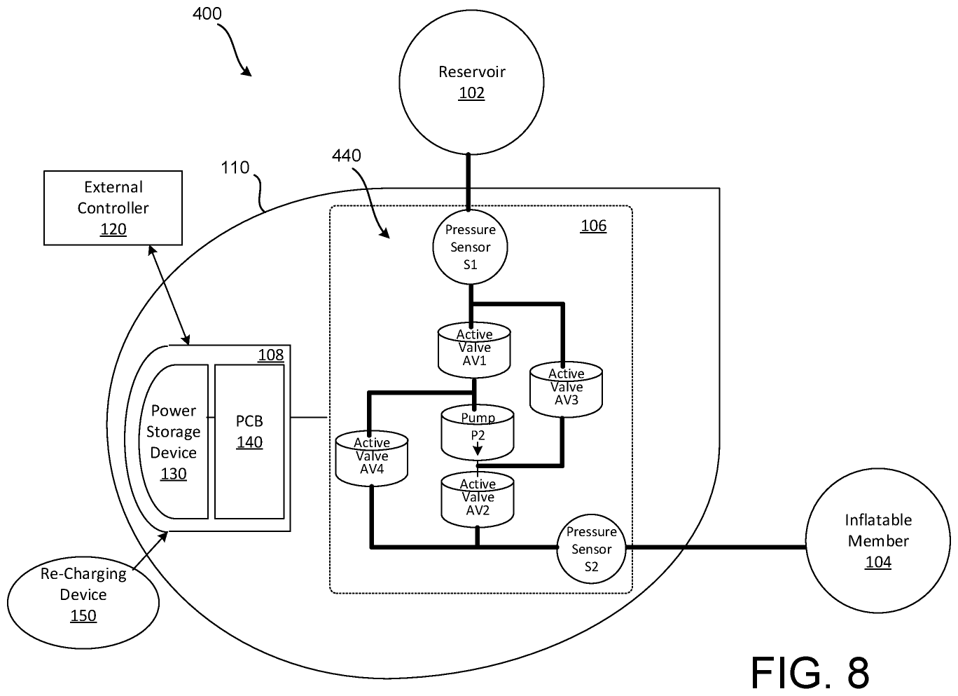

FIG. 8 illustrates the example electronic fluid control system 400 including the fluid control system 106 having a fourth example fluidic architecture 440. The fourth example fluidic architecture 440 employs one pump P2 and four active valves AV1, AV2, AV3 and AV4 to transfer fluid between the reservoir 102 and the inflatable member 104. In an example in which the active valves are piezoelectric valves, the first, second, third and fourth active valves AV1, AV2, AV3, AV4 may be actively and selectively opened and closed in response to selective application of voltage. By actively opening the first active valve AV1 and the second active valve AV2, and actively closing the third active valve AV3 and the fourth active valve AV4, fluid can be pumped from the reservoir 102 to the inflatable member 104 to inflate the inflatable member 104. By actively closing the first active valve AV1 and the second active valve AV2, and actively opening the third active valve AV3 and the fourth active valve AV4, fluid can be pumped from the inflatable member 104 to the reservoir 102 to deflate the inflatable member 104.

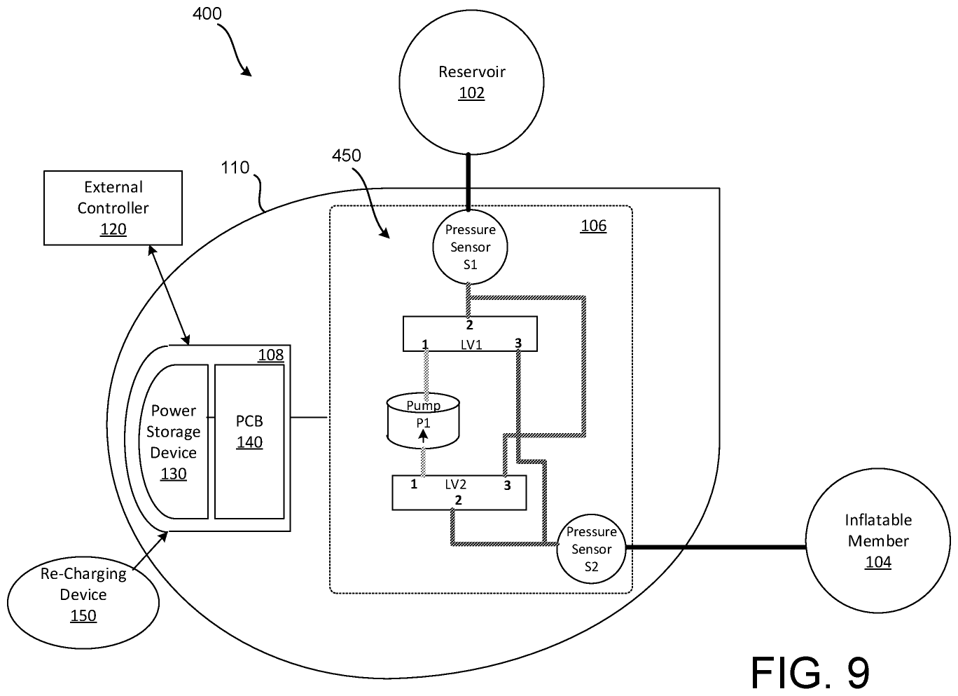

FIG. 9 illustrates the example electronic fluid control system 400 including the fluid control system 106 having a fifth example fluidic architecture 450. The fifth example fluidic architecture 440 employs one pump P1, as did the example fourth fluidic architecture 440. The fifth example fluidic architecture 450 shown in FIG. 9 replaces the four active valves AV1, AV2, AV3, AV4 shown in FIG. 8 with two 3-way latching valves LV1, LV2. In the fifth example fluidic architecture, port 1 on the first latching valve LV1 and port 1 on the second latching valve LV2 are always open. Energizing the first latching valve LV1 allows for one of the other ports 2 or 3 of the first latching valve LV1 to be in communication with the open port 1. Similarly, energizing the second latching valve LV2 allows for one of the other ports 2 or 3 of the second latching valve LV2 to be in communication with the open port 1. By selecting port 2 on both the first latching valve LV1 and the second latching valve LV2, fluid can flow between ports 1 and 2, allowing the pump P1 to transfer fluid from the inflatable member 104 to the reservoir 102, as port 3 of each of the first latching valve LV1 and the second latching valve LV2 is closed. Similarly, by selecting port 3 (and thus closing port 2) of each latching valve LV1, LV2, fluid can flow between ports 1 and 3, allowing the pump to transfer fluid from the reservoir 102 to the inflatable member 104.

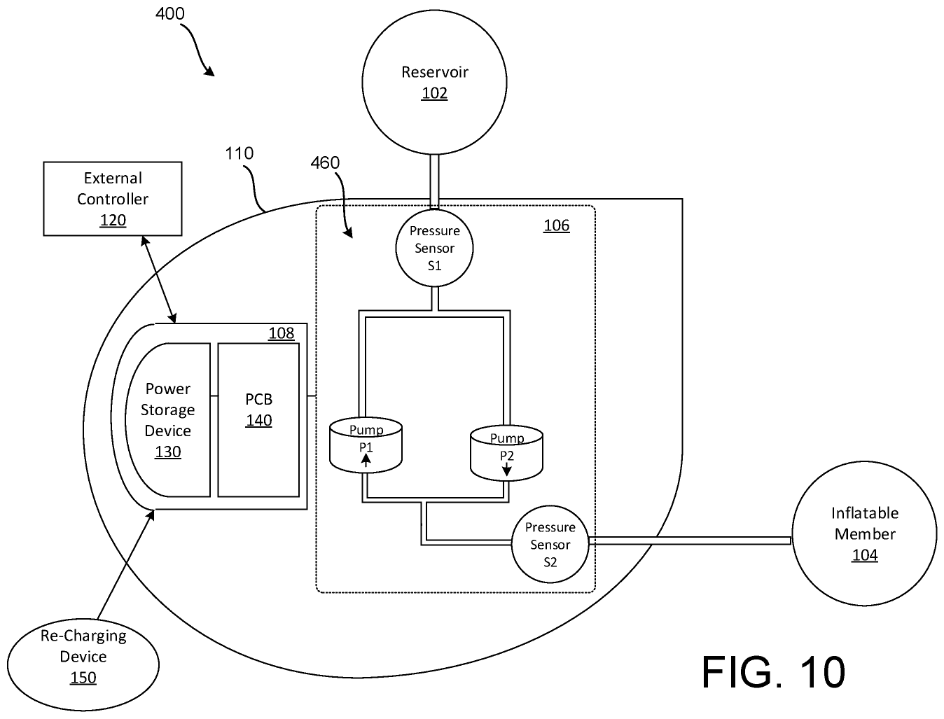

FIG. 10 illustrates the example electronic fluid control system 400 including the fluid control system 106 having a sixth example fluidic architecture 460. The sixth example fluidic architecture includes the first pump P1 generating fluid flow in the first direction, from the inflatable member 104 to the reservoir 102, and the second pump P2 generating fluid flow in the second direction, from the reservoir 102 to the inflatable member 104. In the sixth example fluidic architecture 460 shown in FIG. 10, the first pump P1 and the second pump P2 are combination pump and valve devices. For example, the first pump P1 prevents the flow of fluid through the first fluid channel when the first pump P1 is in the standby mode, and thus not operational/not pumping. Similarly, the second pump P2 prevents the flow of fluid through the second fluid channel when the second pump P2 is in standby mode, and thus not operational/not pumping.

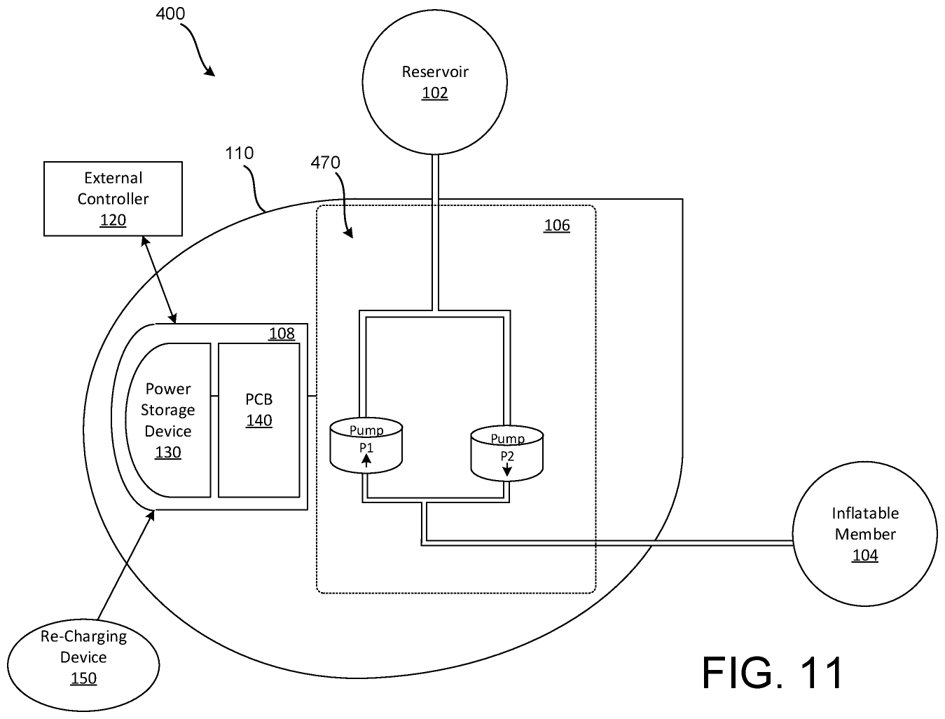

FIG. 11 illustrates the example electronic fluid control system 400 including the fluid control system 106 having a seventh example fluidic architecture 470. The seventh example fluidic architecture includes the first pump P1 generating fluid flow in the first direction, from the inflatable member 104 to the reservoir 102, and the second pump P2 generating fluid flow in the second direction, from the reservoir 102 to the inflatable member 104. In the seventh example fluidic architecture 470 shown in FIG. 11, the first pump P1 and the second pump P2 are combination pump and valve devices, as in the sixth example fluidic architecture 460 shown in FIG. 10, and thus may selectively restrict flow through the fluid channels between the reservoir 102 and the inflatable member 104, in addition to generating fluid flow through the fluid channels. However, in the seventh example fluidic architecture 470 shown in FIG. 11, the first and second pumps P1, P2 may be piezoelectric pumps. Piezoelectric elements of the piezoelectric pumps can sense changes in pressure. Thus, in the seventh example fluidic architecture 470, the first and second pumps P1, P2 (in the form of piezoelectric pumps) can also function as pressure sensing devices, and thus the sensing devices S1, S2 shown in the previous fluidic architectures may be eliminated. This may simplify the fluidic architecture of the fluid control system 106, and may decrease an overall size of the electronic fluid control system 400.

Thus, in some examples, one or more of the valves included in the fluidic architecture of the fluid control system 106 can be piezoelectric valves. Piezoelectric materials produce electrical energy when subjected to mechanical deformation of strain. Conversely, piezoelectric materials are deformed in response to application of an electrical field. That is, piezoelectric materials can convert charge to movement, and can convert movement to charge. These properties allow mechanical valves to be electronically controlled through the application of voltage to the valves. During operation, the fluid operated inflatable device 100 can be subjected to, or experience, external stimuli such as vibration. The source of the vibration can be, for example, vibration generated due to operation of one of the pumps, vibration generated due to the movement of fluid through the fluid operated inflatable device 100, movement and/or other physical activity of the user, and other such sources, both internal and external to the device 100. Given the capability of the piezoelectric material of a piezoelectric valve to generate an electric potential in response to forced movement, these external stimuli can be converted into energy. In some examples, the external stimuli, for example, in the form of vibration, can be converted into energy by the pump that is in the standby mode at the time at which the vibration is experienced.

As described above with respect to the example fluidic architecture shown in FIG. 3, operation of the first pump P1 (with the first valve V1 open, the second pump P2 in standby mode, and the second valve V2 closed) generates fluid flow in the first direction (from the inflatable member 104 towards the reservoir 102) for the deflation of the inflatable member 104. Operation of the second pump P2 (with the second valve V2 open, the first pump P1 in standby mode, and first valve closed) generates fluid flow in the second direction (from the reservoir 102 toward the inflatable member 104) for the inflation of the inflatable member 104. To maintain a set state of the fluid operated inflatable device 100 (i.e., the inflated state or the deflated state) the first and second pumps P1, P2 are in standby mode, and the first and second valves V1, V2 are closed.

In the example fluidic architecture shown in FIG. 3, at least one of the pumps P1, P2 will be in the standby mode at any given time, and thus available to collect the stimuli as described above and convert that stimuli to electrical displacement. In this example, one of the pumps P1 or P2 (the pump that is operational) acts as an energy actuator, or generator, and the other of the pumps P1 or P2 (the pump that is in the standby mode) acts as an energy harvester, or collector. The example fluidic architecture shown in FIG. 3 can include as many as four piezoelectric elements, if the pumps P1, P2 are piezoelectric pumps and the valves V1, V2 are piezoelectric valves. However, in this example, the pumps P1 and P2 act as the actuator and harvester, so that the latching and/or sealing capability of the valves V1, V2 is not compromised during operation of the fluid operated inflatable device 100.

As noted above, during operation of the device 100 in the deflation mode, vibration generated due to operation of the first pump P1 can be transmitted from the first pump P1 to the second pump P2, for example through the manifold in which the fluidics architecture is housed. In this scenario, the piezoelectric element of the second pump P2 (in standby mode) would be ready to harvest the energy generated by the vibration, experienced as movement at piezoelectric element of the second pump P2. In some situations, hydraulic pressure may also act on the second pump P2, thus contributing to the amplitude of the movement experienced by the piezoelectric element of the second pump P2 and causing additional energy to be generated by the amplified movement. During operation in the inflation mode, in which the second pump P2 is operational and the first pump P1 is in the standby mode, the second pump P2 will operate to transfer fluid from the reservoir 102 to the inflatable member 104, and the first pump P1 will harvest the energy produced as a result of the operation of the second pump P2. In some situations, physical movement of the user can translate to movement of the piezoelectric element of the pumps P1, P2. This movement could also be harvested by the first pump P1 and/or the second pump P2 when in the standby mode.

The harvesting and storage of energy in this manner converts energy which would otherwise be dissipated through the device 100, and go unused. Thus, the harvesting and storage of energy in this manner may increase longevity of the power storage device 130, and may increase operating time of the fluid operated device 100 without re-charging or power source replacement. This may also allow for the use of a smaller power storage device 130, thus decreasing an overall size of the electronic fluid control system 400.

As described above, in some examples, the fluid operated inflatable device 100 (for example, in the form of the artificial urinary sphincter 100A or the inflatable penile prosthesis 100B described above) may be electronically controlled by the electronic control system 108. The electronic control system 108 can communicate with an external controller 120 that can be, for example, operated by the user. The external controller 120 can receive user input and transmit the user input to the electronic control system 108 for control of the fluid operated inflatable device 100. The electronic control system 108 can communicate information to the external controller 120 such as, for example, device operating status, system alerts, operating conditions and the like, for consumption by the user. Rapid, reliable communication between the external controller 120 and the electronic control system 108 facilitates the proper functionality and operation of the device 100 in different conditions, providing comfort and ease of use for the patient during the life of the fluid operated inflatable device 100. Rapid, reliable communication between the external controller 120 and the electronic control system 108 can enhance patient safety, and allow the fluid operated inflatable device 100 to adapt to changing conditions, and to employ failsafe measures with or without patient and/or physician intervention.

In some examples, the external controller 120 includes a fob that is tailored specifically for control, monitoring and interaction with the fluid operated inflatable device 100. In some examples, the external controller 120 can be incorporated into an external electronic device that is capable of communication with the electronic control system 108 of the fluid operated inflatable device 100. For example, the external controller 120 can be implemented in an application that is executed by an electronic device such as a smartphone, a tablet computing device and the like.

In some situations, communication between the external controller 120 and the electronic control system 108 of the fluid operated inflatable device 100 may be initiated by the patient, and changes in the operation and control of the fluid operated inflatable device 100 may be initiated manually. In some situations, electronic control of the fluid operated inflatable device 100 is carried out automatically, under the control of the electronic control system 108.

In some examples, manual control of the fluid operated inflatable device 100 may allow the patient to manually configure settings. For example, in some situations, the patient may find that more or less pressure at the inflatable member 104 may improve comfort and/or operability and/or safety. For example, in the case of the example artificial urinary sphincter 100A, the patient may use the external controller 120 to configure a pressure setting at the inflatable cuff 104A based on observed device performance, physical activities and the like. For example, if the patient experiences slight incontinence at a current setting, the patient may use the external controller 120 to increase an occlusion pressure setting on the inflatable cuff 104A. In some examples, the patient may want to adjust the pressure setting at the inflatable cuff 104A due to particular physical activities which may affect continence (for example temporarily, during the physical activity), and may set the adjusted occlusion pressure on the inflatable cuff 104A for a set time period using the external controller 120, allowing the device 100 to revert back to previously stored settings after the set time period has elapsed.

In some examples, manual control of the fluid operated inflatable device 100 can be activated by sub-audible signaling from the patient. In some examples, the sub-audible signaling can be detected by the external controller 120 and transmitted to the electronic control system 108 for control of the fluid operated inflatable device 100. In some examples, the audible signaling can be detected by the electronic control system 108. In some examples, manual control of the fluid operated inflatable device 100 can be activated in response to pressure spikes detected due to tapping, for example sequential tapping implemented by the patient and detected by the fluid operated inflatable device 100. In a situation in which the external controller 120 is for some reason unavailable to the patient (misplaced, not charged, inoperable, and the like), the fluid operated inflatable device 100 may respond to sub-audible signaling from the patient to, for example, adjust a pressure at the inflatable member 104. This may enhance patient safety and comfort.

In some examples, a configurable number of taps, for example, on the torso of the patient, at or near the implanted location of the fluid operated inflatable device, or other location, may define a unique sequence or pattern which triggers manual control of the fluid operated inflatable device 100. This unique sequence or pattern may prevent accidental activation of the fluid operated inflatable device 100 due to inadvertent taps that are detected by the device 100. In some examples, piezoelectric elements of the pumps or valves can act as microphones that can detect a set audible or sub-audible signal. In some examples, the detected signal can, for example, command the pump or valve to open, with the corresponding displacement generating a measurable current.

In some examples, one or both of the implantable fluid operated inflatable device 100 and/or the external controller 120 include a motion detecting device such as, for example, an accelerometer, that can detect a motion event. In some situations, motion events can cause a change in conditions within the fluid operated inflatable device 100 that may benefit from an adjustment to the operating parameters of the device 100 for the motion event. For example, in the artificial urinary sphincter 100A described above, motion related to events such as coughing, sneezing, lifting, exercise/physical activity, and the like may lead to incontinence. Detection of this type of motion event by the accelerometer may trigger the execution of an algorithm, for example, by the processor of the electronic control system 108, that increases a pressure at the inflatable cuff 104A to provide extra pressure at the urethra during the motion event to prevent incontinence. In some examples, the need for additional pressure at, for example the inflatable member 104, in response to these types of motion events may be detected based on changes in pressure/pressure fluctuations detected by the sensing devices included in the fluidics architecture. For example, a detected increase in intra-abdominal pressure (due to, for example, a compression due to a cough or sneeze, a bending and/or lifting motion and the like) can be conveyed to the reservoir 102, thus increasing the internal pressure of the device 100 at the reservoir 102. The increased pressure at the reservoir 102 detected by one of the sensing devices can be processed an algorithm executed by the electronic control system, so that operation of the pumps and valves within the fluidics system can be adjusted to apply appropriate pressure at the reservoir 102 and the inflatable member 104 to maintain a current state of the fluid operated inflatable device 100.

In some examples, manual control of the fluid operated inflatable device 100 in a situation in which the external controller 120 is for some reason unavailable to the patient (misplaced, not charged, inoperable, and the like) may be implemented by the use of a backup activation device such as a magnet. For example, in a situation in which the external controller is unavailable and the patient needs to release pressure on the inflatable cuff 104A of the artificial urinary sphincter 100A, application of the backup activation device/magnet at a position corresponding to the implanted device 100A may activate a read switch, controlling the pumps and valves within the fluidic architecture to operate to release pressure on the inflatable cuff 104A, allowing the inflatable cuff 104A to open and release the urethra.

In some examples, manual control of the fluid operated inflatable device 100, particularly when the external controller 120 is unavailable, may include manual pressure that is externally applied to the device 100. In some examples, this may include a first sequence of externally applied pressures that acts as a wake-up signal, followed by a second sequence of externally applied pressures that serves as an activation signal. For example, the externally applied pressure may be in the form of tugs on the penis, which generate pressure fluctuations in the fluid channels of the artificial urinary sphincter 100A, particularly in the vicinity of the inflatable cuff 104A. In this example, a first sequence of tugs may wake the artificial urinary sphincter 100A, and a second sequence of tugs may signal a release of the pressure at the inflatable cuff 104A, so that the cuff opens to release the urethra and the patient is able to void. In some examples, pressure in the form of tugging in this manner can also generate a sub-audible signal that can be detected by the piezoelectric elements of the pumps and valves acting as microphones, as described above.

As noted above, in some situations, electronic control of the fluid operated inflatable device is carried out automatically under the control of the electronic control system 108. This may allow for substantially continuous system monitoring, diagnostics and adjustment, and for the output of alerts in response to detection of conditions requiring intervention by the patient and/or physician.

In some examples, the electronic control system 108 can monitor operation of the fluid operated inflatable device 100 to detect conditions which may be indicative of leakage, blockage and the like which may compromise operation of the device 100 and/or lead to failure of the device 100. For example, the electronic control system 108 can monitor an amount of time to reach a certain pressure at a certain position within the fluidic architecture. A change in pumping time, for example, in excess of a set threshold or a set range, and/or an inability to reach a certain pressure or a certain pressure range may be indicative of a leak or a blockage within the fluid channels of the fluid operated inflatable device 100. In some examples, the electronic control system 108 generates an alert, for example for output through the external controller 120, alerting the patient and/or the physician to a possible condition which may compromise operation of the device 100 and/or which may lead to failure of the device 100. In some examples, the electronic control system 108 can control operation of the pumps and valves so that fluid is sealed within portions of the device 100 that are not experiencing leakage.

In some examples, automatic control of the fluid operated inflatable device 100 includes collection and storage of data for diagnosis by the physician and adjustment of patient care protocols. In the case of the artificial urinary sphincter 100A, diagnosis often relies on a bladder diary completed manually by the patient. In some examples, the electronic control system 108 of the artificial urinary sphincter 100A can measure and record a number of times in a day the patient must void, a start to finish time for each void event, and other such data. In some examples, the elapsed time for each void event may be determined based on an amount of open time of the inflatable cuff 104A and/or an amount of closed time of the inflatable cuff 104A. In some examples, acoustic properties of the piezoelectric elements of the pumps and/or valves may be used to calculate start and finish times for each void event. Data collected and tracked in this manner may be used by the physician for follow on diagnosis and treatment.

In some examples, automatic control of the fluid operated inflatable device 100 includes the automatic control of pressure at the inflatable member 104 and/or the reservoir 102 in response to certain conditions. For example, the electronic control system 108 may detect that there has been no communication from the external controller 120 to the implanted fluid operated inflatable device 100 for a set period of time (indicating the external controller 120 is for some reason unavailable or inoperable), and/or the inflatable member 104 has been in the inflated condition for greater than a set period of time, and the like. In response to detection of this type of condition, the electronic control system 108 may relieve a pressure setting within the implanted fluid operated inflatable device 100, to for example, relieve the pressure at the inflatable member 104, as a failsafe measure.

In some examples, automatic control of the fluid operated inflatable device 100 can provide for the detection of infection. Sensing devices, such as one or more thermocouples in the device 100, can record temperatures that are indicative of internal body temperature of the patient. These temperatures can be stored, for example, in the memory of the electronic control system 108. Sensed temperatures, and fluctuation and/or increases in sensed temperatures, can provide an early indication of infection. In some examples, this early prediction of infection can trigger an alert to be output to the user through the external controller 120, for treatment by the physician.

In some examples, automatic control of the fluid operated inflatable device 100 can provide for the correction of internal device pressures based on atmospheric, or barometric, pressures detected by an external device, such as the external controller 120, and transmitted to the electronic control system 108. Identification of the atmospheric pressure (and changes in atmospheric pressure), in some examples essentially in real time, allow the electronic control system 108 to automatically control the operation of the pumps and valves to adjust internal pressures of the device 100 based on the detected atmospheric pressure. The ability to automatically adjust device operation to account for changes in atmospheric pressure may ensure that the implanted fluid operated inflatable device 100 maintains the correct internal pressures even in the event of changing atmospheric conditions.

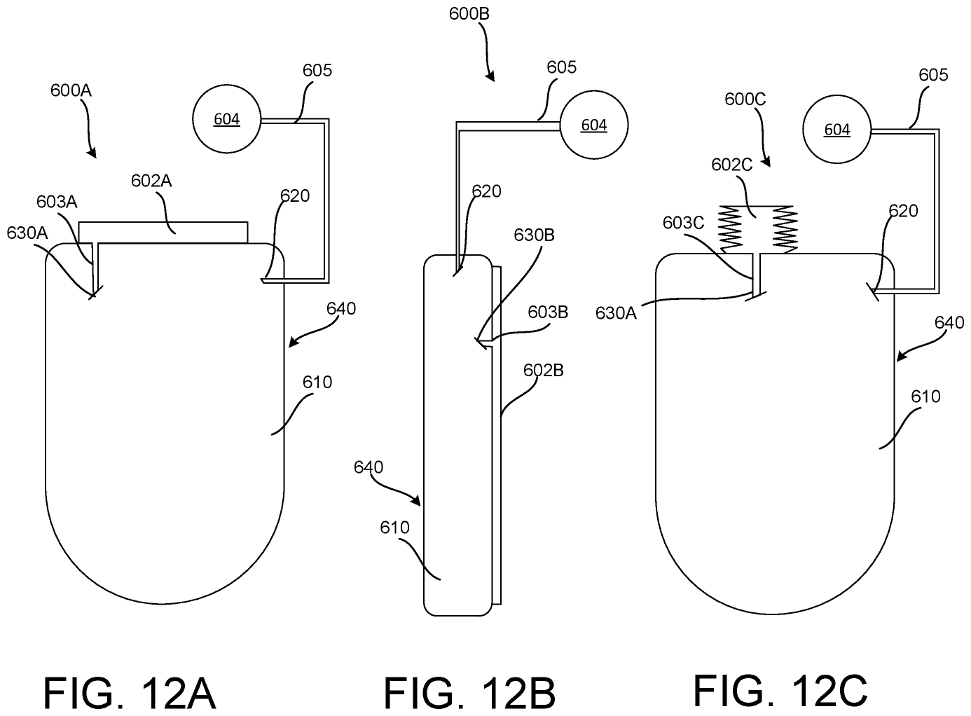

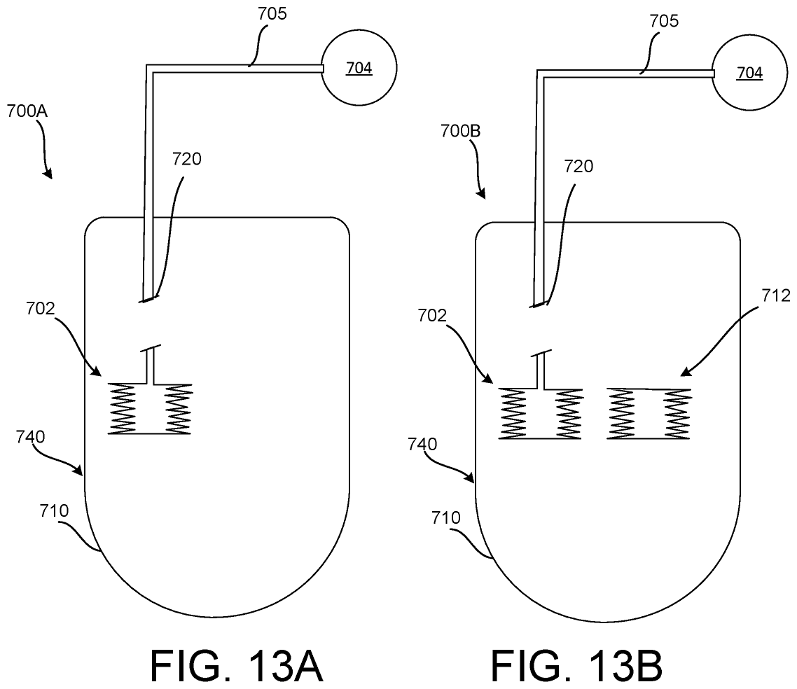

The example implantable fluid operated inflatable device 100 described above (in the form of, for example, the artificial urinary sphincter 100A and/or the inflatable penile prosthesis 100B) includes the fluid reservoir 102 connected to the inflatable member 104 by the electronic fluid control system 400 by fluid conduits 103, 105, to provide for the transfer of fluid between the reservoir 102 and the inflatable member 104. FIGS. 12A-1C illustrate example implantable fluid operated inflatable devices in which the fluid reservoir is coupled to a housing of the electronic fluid control system. FIGS. 13A and 13B illustrate example implantable fluid operated inflatable devices in which the fluid reservoir is received within the housing of the electronic fluid control system.

FIGS. 12A-12C are schematic illustrations of example implantable fluid operated inflatable devices 600. In particular, FIG. 12A is a schematic illustration of a first example implantable fluid operated inflatable device 600A, FIG. 12B is a schematic illustration of a second example implantable fluid operated inflatable device 600B, and FIG. 12C is a schematic illustration of a third example implantable fluid operated inflatable device 600C. Each of the three example fluid operated inflatable devices 600A, 600B, 600C shown in FIGS. 12A-12C includes an inflatable member 604 coupled to an electronic fluid control system 640 by a fluid conduit 605, and a fluid reservoir 602 coupled, for example, directly coupled, to a housing 610 of the electronic fluid control system 640.

The example electronic fluid control system 640 may include components included in the example electronic fluid control system 400 described above with respect to FIGS. 5-11, including for example, the power storage device 130, PCB 140 of the electronics control system 108, and fluid control system 106 including the example fluidic architectures received in the housing 110, as described above with respect to FIGS. 5-11. The principles to be described with respect to the example implantable fluid operated inflatable devices 600A, 600B, 600C may be applied to various different types of implantable fluid operated inflatable devices, including for example the artificial urinary sphincter 100A and the inflatable penile prosthesis 100B described above.

The example fluid operated inflatable device 600A shown in FIG. 12A includes the electronic fluid control system 640 including electronic components and fluidics components as described above, received in a hermetic housing 610. A fluid conduit 605 has a first end coupled to the inflatable member 604, and a second end extending through a port 620 formed in the housing 610 for connection to the fluid control system received in the housing 610, to provide for the transfer of fluid to and from the inflatable member 704. In the arrangement shown in FIG. 12A, the reservoir 602A is coupled to a top surface portion of the hermetic housing 610 (in the example orientation shown in FIG. 12A), or a transverse plane of the hermetic housing 610. In some examples, the reservoir 602A is fixed, for example, adhered or bonded, to the housing 610. A fluid conduit 603A has a first end connected to the reservoir 602A, and a second end that extends through a port 630A in the housing 610 for connection to the fluid control system received in the housing 610, to provide for the transfer of fluid to and from the reservoir 602A. This example arrangement may present a smaller mating surface area between the hermetic housing 610 and the reservoir 602A and may expose the reservoir 602A to less pressure due to patient movement (than, for example, the example arrangement shown in FIG. 12B). In some examples, lattice (not shown in FIG. 12A) may be placed surrounding the exterior of the reservoir 602A, to prevent exertion of external pressure on the reservoir 602A.

The example fluid operated inflatable device 600B shown in FIG. 12B includes the electronic fluid control system 640 including the inflatable member 604 connected to the fluid control system received in the hermetic housing 610 via the fluid conduit 605 as described above. The example fluid operated inflatable device 600B includes a reservoir 602B coupled to a side portion of the hermetic housing 610 (in the example orientation shown in FIG. 12B), or a coronal plane of the housing 610. In some examples, the reservoir 602B is fixed, for example, adhered or bonded, to the housing 610. A conduit 603B has a first end connected to the reservoir 602B, and a second end that extends through a port 630B in the housing 610 for connection to the fluid control system received in the housing 610, to provide for the transfer of fluid to and from the reservoir 602B. In the example arrangement shown in FIG. 12B, the reservoir 602B is bonded to the largest surface of the housing 610. The larger surface area of the of the reservoir 602B may reduce the expansion required of the reservoir 602B (compared to the example arrangement shown in FIG. 12A).

The example fluid operated inflatable device 600C shown in FIG. 12C includes the electronic fluid control system 640 including the inflatable member 604 connected to the fluid control system received in the hermetic housing 610 via the fluid conduit 605 as described above. The example fluid operated inflatable device 600C includes a reservoir 602C having a bellows structure coupled to a top portion of the hermetic housing 610 (in the example orientation shown in FIG. 12C). In some examples, a portion, for example, a bottom portion of the reservoir 602C is fixed, for example, adhered or bonded, to the housing 610, allowing the remaining portion of the bellows structure forming the reservoir 602C to expand and contract. A conduit 603C has a first end connected to the reservoir 602C, and a second end that extends through a port 630C in the housing 610 for connection to the fluid control system received in the housing 610, to provide for the transfer of fluid to and from the reservoir 602C. The bellows structure of the example reservoir 602C shown in FIG. 12C contracts as fluid is expelled from the reservoir 602C, and expands as fluid flows into the reservoir 602C. The bellows structure of the example reservoir 602C shown in FIG. 12C allows for a wider range of materials to be used for the reservoir 602C, including for example a titanium polymetric material which would allow the reservoir 602C to be hermetically sealed to the hermetic housing 610. In some examples, lattice (not shown in FIG. 12C) may be placed surrounding the exterior of the reservoir 602C, to prevent exertion of external pressure on the reservoir 602C.

The two-piece example fluid operated inflatable devices 600A, 600B, 600C including external fluid reservoirs 602A, 602B, 602C attached to the hermetic housing 610 allows for expansion and contraction of the reservoirs 602A, 602B, 602C outside of the hermetic housing 610 with limited resistance, while reducing the overall device 600 to two components (i.e., the inflatable member 604 and the housing 610 having the reservoir 602 attached thereto). In some situations, this design may reduce surgical procedure time and complexity. In some situations, this design may allow for the hermetic housing 610 to be sutured in place within the patient, thus reducing in-vivo drift during the life of the implanted fluid operated inflatable device 600.

FIGS. 13A and 13B are schematic illustrations of example implantable fluid operated inflatable devices 700. In particular, FIG. 13A is a schematic illustration of a first example implantable fluid operated inflatable device 700A, and FIG. 13B is a schematic illustration of a second example implantable fluid operated inflatable device 700B. The example fluid operated inflatable devices 700A, 700B shown in FIGS. 13A and 13B each include an inflatable member 704 coupled to an electronic fluid control system 740 by a fluid conduit 705, and a fluid reservoir 702 received within a hermetic housing 710 of the electronic fluid control system 740.

The example electronic fluid control system 740 may include components included in the example electronic fluid control system 400 described above with respect to FIGS. 5-11, including for example, the power storage device 130, PCB 140 of the electronics control system 108, and fluid control system 106 including the example fluidic architectures received in the housing 110, as described above with respect to FIGS. 5-11. The principles to be described with respect to the example implantable fluid operated inflatable devices 700A and 700B may be applied to various different types of implantable fluid operated inflatable devices, including for example the artificial urinary sphincter 100A and the inflatable penile prosthesis 100B described above.

The example fluid operated inflatable device 700A shown in FIG. 12A includes the electronic fluid control system 740 including electronic components and fluidics components as described above, received in a hermetic housing 710. A fluid conduit 705 has a first end coupled to the inflatable member 704, and a second end extending through a port 720 formed in the housing 710 for connection to the fluid control system received in the housing 710, to provide for the transfer of fluid to and from the inflatable member 704. In the arrangement shown in FIG. 13A, the reservoir 702A is received within the hermetic housing 710. Because the environment within the hermetic housing 710 holds a fixed amount of gas/fluid, changes in volume within the reservoir 702 will cause a change in pressure within the hermetic housing 710, limiting the amount by which the reservoir 702 can expand and contract within the hermetic housing 710. The use of a bellows structure for the reservoir 702 may alleviate this, particularly if the hermetic housing 710 is filled with a gas that is relatively easily compressed, such as, for example helium or argon.

The example fluid operated device 700B shown in FIG. 12B includes a closed bellows 12 within the hermetic housing 710. The closed bellows 712 may be filled with a compressible fluid, acting as a sacrificial gas, allowing the closed bellows 712 to expand as the reservoir 702 contracts, and to contract as the reservoir 702 expands. That is, the reservoir 702 (having the bellows structure) expands as fluid is introduced into the reservoir 702, and the closed bellows 712 contracts in response to the expansion of the reservoir 702. The reservoir 702 contracts as fluid is expelled from the reservoir 702, and the closed bellows 712 expands in response to the contraction of the reservoir 702.

The example two-piece fluid operated inflatable devices 700A, 700B including an internal fluid reservoir 702 installed within the hermetic housing 610 may reduce an overall size of the implanted fluid operated inflatable device 700. In some situations, this design may reduce surgical procedure time and complexity. In some situations, this design may allow for the hermetic housing 710 to be sutured in place within the patient, thus reducing in-vivo drift during the life of the implanted fluid operated inflatable device 700.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated inflatable device, comprising:
a fluid reservoir;
an inflatable member;
an electronic fluid control system coupled between the fluid reservoir and the inflatable member and configured to control fluid between the fluid reservoir and the inflatable member, the electronic fluid control system including:
a housing;
a fluid control system received in the housing, including fluidic architecture including pumping device positioned in a fluid passageway within in the housing;
an electronic control system received in the housing, the electronic control system including:
at least one processor configured to control operation of the at least one pump and at least one valve; and
a communication module configured to communicate with at least one external device; and
at least one pressure sensing device configured to sense a fluid pressure in the implantable fluid operated inflatable device, and to transmit the sensed pressure to the electronic control system.

2. The implantable fluid operated inflatable device of claim 1, wherein the reservoir is bonded to an outer surface of the housing.

3. The implantable fluid operated inflatable device of claim 1, wherein the reservoir includes a bellows structure configured to contract as fluid is expelled from the reservoir, and to expand as fluid flows into the reservoir.

4. The implantable fluid operated inflatable device of claim 3, wherein the reservoir is received within the housing.

5. The implantable fluid operated inflatable device of claim 4, further comprising a closed bellows within the housing, wherein the closed bellows is filled with a compressible fluid, such that the closed bellows is configured to contract in response to expansion of the reservoir, and to expand in response to contraction of the reservoir.

6. The implantable fluid operated inflatable device of claim 1, wherein the electronic control system is configured to receive a user input from the external device, and to control operation of the at least one pumping device in response to the received user input.

7. The implantable fluid operated inflatable device of claim 6, wherein the electronic control system is configured to adjust operation of the at least one pumping device to reduce a pressure at the inflatable member and initiate deflation of the inflatable member in response to detection of a signal generated by interaction of a magnet with the electronic control system, positioned corresponding to the fluid-controlled inflatable device for a preset period of time.

8. The implantable fluid operated inflatable device of claim 1, wherein the electronic control system is configured to control operation of the at least pumping device in response to user inputs including at least one of:
a fluctuation in pressure detected by the at least one sensing device in response to a tapping input or a tugging input; or
a motion event detected by a motion detecting device of the fluid operated inflatable device or the external device.

9. The implantable fluid operated inflatable device of claim 8, wherein the tapping input includes a series of taps in a preset sequence detected by a piezoelectric element of the at least one pumping device.

10. The implantable fluid operated inflatable device of claim 9, wherein the preset sequence includes:
a wake-up sequence to wake the fluid operated inflatable device, including a first tapping sequence defined by a first number of taps in a first pattern; and
an activation sequence corresponding to a user input, including a second tapping sequence defined by a second number of taps in a second pattern.

11. The implantable fluid operated inflatable device of claim 1, wherein the electronic control system is configured to monitor pressure levels in the fluid-controlled inflatable device, and to control operation of the at least one pumping device in response to detected fluctuations in pressure, including:
control the at least one pumping device to reduce a pressure at the inflatable member and deflate the inflatable member in response to detection of the inflatable member in an inflated state for greater than a preset period of time;
control the at least one pumping device to maintain a current state of the fluid-controlled inflatable device in response to detection of a spike in pressure having a duration that is less than a preset period of time; and
control the at least one pumping device to maintain the current state of the fluid-controlled inflatable device in response to detection of a change in atmospheric conditions.

12. The implantable fluid operated inflatable device of claim 1, wherein the electronic control system is configured to:
detect a failure in the fluid-controlled inflatable device in response to detection of a time to reach a set pressure exceeding a set period of time or an inability to reach the set pressure;
output an alert of the detected failure to the external device; and
isolate fluid from an area of the detected failure.

13. The implantable fluid operated inflatable device of claim 1, wherein the at least one pumping device includes a first piezoelectric pump in a first fluid channel of the fluidic architecture and a second piezoelectric in a second fluid channel of the fluidic architecture, wherein:
in a deflation mode,
the first piezoelectric pump is configured to operate to pump fluid from the inflatable member to the reservoir, while the second piezoelectric pump is in a standby mode; and
vibration generated by operation of the first piezoelectric pump is harvested by the second piezoelectric pump in the standby mode for conversion to energy; and
in an inflation mode,
the second piezoelectric pump is configured to operate to pump fluid from the reservoir to the inflatable member, while the first piezoelectric pump is in the standby mode; and
vibration generated by operation of the second piezoelectric pump is harvested by the first piezoelectric pump in the standby mode for conversion to energy.

14. The implantable fluid operated inflatable device of claim 13, wherein, in a standby mode of the fluid operated inflatable device in which the first piezoelectric pump and the second piezoelectric pump are both in the standby mode, vibration generated due to motion of a patient in which the fluid operated inflatable device is implanted is harvested by the first piezoelectric pump and the second piezoelectric pump for conversion to energy.

15. The implantable fluid operated inflatable device of claim 1, wherein the fluidic architecture includes:
a first uni-directional pump and a first passive valve positioned in a first fluid passageway to selectively generate and control fluid flow in a first direction, from the inflatable member toward the reservoir;
a second uni-directional pump and a second passive valve positioned in a second fluid passageway to selectively generate and control fluid flow in a second direction, from the reservoir to the inflatable member;
a first sensing device positioned to sense a fluid pressure at the reservoir;
a second sensing device positioned to sense a fluid pressure at the inflatable member; and
an active valve positioned in-line with the inflatable member, wherein,
in a first mode, the active valve is configured to be closed by the electronic control system in response to detection of a pressure spike at the inflatable member to prevent deflation of the inflatable member; and
in a second mode, the active valve is configured to be opened by the electronic control in response to detection of a power loss to the electronic fluid control system to allow deflation of the inflatable member.

16. The implantable fluid operated inflatable device of claim 1, wherein the fluidic architecture includes:
a first uni-directional pump positioned in a first fluid passageway and configured to generate a flow of fluid in a first direction, from the inflatable member toward the reservoir;
a second uni-directional pump positioned in a second fluid passageway and configured to generate a flow of fluid in a second direction, from the reservoir toward inflatable member;
a first passive valve positioned in the first fluid passageway, between the first uni-directional pump and the reservoir so as to restrict fluid flow in the first direction in the first fluid passageway and to prevent back flow of fluid in the first fluid passageway while the second uni-directional pump is in an operational mode and the first uni-directional pump is in a standby mode;
a second passive valve positioned in the second fluid passageway, between the second uni-directional pump and the reservoir so as to restrict fluid flow in the second direction in the second fluid passageway and to prevent back flow of fluid in the second fluid passageway while the first uni-directional pump is in an operational mode and the second uni-directional pump is in a standby mode;
a first sensing device positioned to sense a fluid pressure at the reservoir; and
a second sensing device positioned to sense a fluid pressure at the inflatable member.

17. The implantable fluid operated inflatable device of claim 1, wherein the fluidic architecture includes:
a uni-directional pump positioned in a fluid passageway;
a first active valve positioned in the fluid passageway, between the pump and the reservoir, and configured to be selectively activated by the electronic control system;
a second active valve positioned in the fluid passageway, between the pump and the inflatable member, and configured to be selectively activated by the electronic control system;
a third active valve positioned in a fluid passageway between the pump and the reservoir and configured to be selectively activated by the electronic control system;
a fourth active valve in a fluid passageway between the pump and the inflatable member and configured to be selectively activated by the electronic control system, wherein,
in an inflation mode, the first active valve and the second active valve are opened by the electronic control system and the third active valve and the fourth active valve are closed by the electronic control system so that fluid is pumped from the reservoir to the inflatable member; and
in a deflation mode, the third active valve and the fourth active valve are opened by the electronic control system and the first active valve and the second active valve are closed by the electronic control system so that fluid is pumped from the inflatable member to the reservoir.

18. The implantable fluid operated inflatable device of claim 1, wherein the fluidic architecture includes:
a first combined pump and valve device positioned in a first fluid passageway to selectively generate and control fluid flow in a first direction, from the inflatable member toward the reservoir;
a first sensing device positioned to sense a fluid pressure at the reservoir;
a second combined pump and valve device positioned in a second fluid passageway to selectively generate and control fluid flow in a second direction, from the reservoir toward the inflatable member; and
a second sensing device positioned to sense a fluid pressure at the inflatable member.

19. The implantable fluid operated inflatable device of claim 1, wherein the fluidic architecture includes:
a first piezoelectric pump and valve device positioned in a first fluid passageway, wherein the first piezoelectric pump and valve device is configured to selectively generate and control fluid flow in a first direction, from the inflatable member toward the reservoir, and to sense a fluid pressure at the reservoir; and
a second piezoelectric pump and valve device positioned in a second fluid passageway, wherein the second piezoelectric pump and valve device is configured to selectively generate and control fluid flow in a second direction, from the reservoir toward the inflatable member, and to sense a fluid pressure at the inflatable member.

20. The implantable fluid operated inflatable device of claim 1, wherein the fluidic architecture includes:
a pump;
a first three-way valve positioned between the pump and the reservoir, the first three-way valve having a first port thereof open to maintain fluidic communication with the pump; and
a second three-way valve positioned between the pump and the inflatable member, the second three-way valve having a first port thereof open to maintain fluidic communication with the pump, wherein,
in a deflation mode,
a second port of the first three-way valve is opened and a third port of the first three-way valve is closed to direct fluid flow from first port to the second port of the first three-way valve; and
a second port of the second three-way valve is opened and a third port of the second three-way valve is closed to direct fluid flow from the first port to the second port of the second three-way valve; and
in an inflation mode,
the second port of the first three-way valve is closed and the third port of the first three-way valve is opened to direct fluid flow from first port to the third port of the first three-way valve; and
the second port of the second three-way valve is closed and the third port of the second three-way valve is opened to direct fluid flow from the first port to the third port of the second three-way valve.
